(19) United States
(12) Patent Application Publication
(10) Pub. No.: US 2007/0037848 A1
(43) Pub. Date: Feb. 15, 2007

(54) Treatment of neurological conditions

(76) Inventors: Colin Louis Masters, Victoria (AU); Robert A. Cherny, Victoria (AU)

Correspondence Address: SCULLY, SCOTT, MURPHY & PRESSER, 400 GARDEN CITY PLAZA, SUITE 300, GARDEN CITY, NY, 11530, US

(21) Appl. No.: 10/552,258

(22) PCT Filed: Apr. 2, 2004
(86) PCT No.: PCT/AU04/00427

§ 371 Date: Aug. 25, 2006

Related U.S. Application Data
(60) Provisional application No. 60/460,155, filed on Apr. 3, 2003.

(51) Int. Cl. A61K 31/4709

(52) U.S. Cl. 514/311; 514/312; 514/314

(57) ABSTRACT

The present invention relates generally to the treatment of conditions and/or disorders associated with or exacerbated by oxidative stress and which have symptoms including cognitive impairment such as pre- or mild cognitive impairment or memory loss. The agents and methods useful for the practice of the present invention are proposed to modulate and in particular reduce levels of reactive oxygen species thereby minimizing oxidative stress. The present invention further provides methods and agents for the treatment of and/or prophylaxis of neurological diseases and in particular those associated with or facilitated by oxidative stress. Neurological disorders contemplated herein include any condition leading to cognitive impairment such as pre- or mild cognitive impairment or memory loss. The present invention provides therefore methods and agents for treating, ameliorating the symptoms of and/or otherwise arresting cognitive impairment such as pre- or mild cognitive impairment or memory loss.

### BACKGROUND OF THE INVENTION

1. Field of the Invention

The present invention relates generally to the treatment of conditions and/or disorders associated with or exacerbated by oxidative stress and which have symptoms including cognitive impairment such as pre- or mild cognitive impairment or memory loss. The agents and methods useful for the practice of the present invention are proposed to modulate and in particular reduce levels of reactive oxygen species thereby minimizing oxidative stress. The present invention further provides methods and agents for the treatment of and/or prophylaxis of neurological diseases and in particular those associated with or facilitated by oxidative stress. Neurological disorders contemplated herein include any condition leading to cognitive impairment such as pre- or mild cognitive impairment or memory loss. The present invention provides therefore methods and agents for treating, ameliorating the symptoms of and/or otherwise arresting cognitive impairment such as pre- or mild cognitive impairment or memory loss.

2. Description of the Prior Art

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

Scientists have unearthed evidence suggesting that the onset and progression of Parkinson's disease and other neurological diseases, such as Alzheimer's disease, could be due to oxidative stress. This association was made based on the discovery that alpha-synuclein (α-syn), a protein found in the presynaptic terminal of neurons is the main component of several markers of neurological disorders, including Lewy bodies (LB) and Lewy neurites (LN), both characteristic lesions of Parkinson's disease. It was shown that the α-syn present in the lesions was nitrated, a sign of oxidative stress caused by free radical damage.

The incidence of neurological diseases such as Parkinson's disease and Alzheimer's are continuing to increase as a result of our aging population.

The life span is thought to be biologically fixed for each species, and the length of the human life span is uncertain, but may be up to 120 years. Since life expectancy has risen significantly in this century, the elderly are an increasing segment of our population, and their health care needs will continue to grow for decades.

Although normal aging is characterized by modest reductions in the mass and volume of the human brain, which may be due to the atrophy and/or death of brain cells, these changes are far more profound in the brains of patients who succumb to a neurological condition. Most of these conditions are sporadic (i.e. not due to genetic mutations) and of unknown cause, but hundreds of different mutations in many genes have been shown to cause familial (inherited) variants of several neurological conditions. Many of the dozen or more genes that harbor these mutations were discovered in the quest to determine the genetic basis of neurological conditions just in the last ten years. Neurological conditions evolve gradually after a long period of normal brain function, due to progressive degeneration (i.e. nerve cell dysfunction and death) of specific brain regions. Since symptomatic expression of disease occurs when nerve cell loss exceeds a “threshold” for the continuing function (e.g. memory, movement) performed by the affected brain region, the actual onset of brain degeneration may precede clinical expression by many years.

Intellectual and higher integrative cognitive faculties become progressively impaired and interfere with activities of daily living in neurological conditions resulting in dementia. The precise prevalence of dementia in the elderly population is unknown, but may be 15% of people over 65 years old with 5% severely and 10% mildly to moderately demented. The prevalence of severe dementia increases from 1% at 65 years to 45% at 85 years. There are many causes of dementia, but Alzheimer's Disease (AD) accounts for 50% of demented patients over 65 years of age.

Amyloid plaques are known to be present in the brains of individuals with certain neurological diseases, but it is not known whether it is symptomatic of an underlying disease process, or is actually involved in the aetiology of the disease. For example, some authors believe that the Aβ deposits may be indicative of a normal brain defence mechanism, in which the brain attempts to sequester the Aβ; such deposits can be present in the brains of normal individuals. There is a mutation of tau protein in which neurofibrillary tangles, but no amyloid plaques are present in the brain; this condition is known as tauopathy.

It has also been suggested that deposition of amyloid-like fibrils may also be important in other neurological diseases, in which α-synuclein fibrils are deposited. These include Parkinson's disease, dementia with Lewy body formation, multiple system atrophy, Hallerboden-Spatz disease, and diffuse Lewy body disease.

One of the competing theories of the aetiology of AD is that the causative step(s) lies within the pathway of the intracerebral biogenesis and accumulation of the Aβ amyloid protein (see recent reviews by Selkoe, Physiol Rev 81(2): 741-766, 2001; Beyreuther et al., Springer, Berlin, 2001; Bush, Science 292: 2251-2252, 2001). However, to date, no drugs or agents which target this pathway have been demonstrated to have a lasting effect on treating a neurological disease and/or ameliorating the effects of cognitive impairment or memory loss especially when caused by oxidative stress.

Accordingly, until the advent of the present invention there have been no therapeutic protocols developed which address the symptoms of cognitive impairment or memory loss especially when induced by oxidative stress.

Given the increasing prevalence of psychologically and clinically debilitating conditions caused by oxidative stress, there is clearly a need to find more efficacious ways to treat and, preferably, prevent the onset of the symptoms of such conditions including cognitive impairment memory loss.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In accordance with the present invention, it is determined that a range of conditions or disorders resulting in symptoms such as cognitive impairment or memory loss are associated with oxidative stress. Particularly important conditions in this regard include or are caused by neurological conditions and disorders are associated with oxidated stress or other neurological conditions associated with reactive oxygen species. Accordingly, the present invention provides methods and agents for reducing reactive oxygen species' formation and hence provide a treatment or prophylaxis for any condition associated with oxidative stress especially those having symptoms of cognitive impairment or memory loss. Even more particularly the conditions and disorders contemplated herein are neurological conditions which contribute to cognitive impairment such as pre- or mild cognitive impairment or memory loss. Accordingly, the present invention further provides methods and agents for ameliorating the symptoms of and/or otherwise arresting cognitive impairment such as pre- or mild cognitive impairment or memory loss.

The present invention provides, therefore, agents which modulate levels of active oxygen species thereby minimizing oxidative stress for use in the treatment and prophylaxis of cognitive impairment such as pre- or mild cognitive impairment or memory loss resulting from oxidative stress. Particularly important disorders contemplated by the present invention are neurological diseases resulting from oxidative stress such as Alzheimer's disease, dementia associated with Down's syndrome, Creutzfeldt-Jakob disease, dementia associated with and Parkinson's disease and neurological disease arising from oxidative stress resulting from diabetes, stroke and other cardiovascular assaults. The agents are conveniently in the form of a composition comprising the agent and one or more pharmaceutically acceptable carriers, diluents and/or excipients. As a result of administration of an agent or composition of the present invention to a subject suffering from or having a pre-disposition to suffer from a neurological disorder or other disorder caused by or exacerbated by oxidative stress.

Reference herein to the treatment and prophylaxis of neurological disorders includes effecting an improvement in a subject's cognitive or memory function.

In a preferred embodiment, the agent is a metal binding agent, the administration of which results in decreased levels of reactive oxygen species relative to the level prior to treatment resulting in improved cognitive function. Reference to “administration” includes reference to delivering an agent.

In a related embodiment, the agent is a metal binding agent, the administration of which, results in the elevation of a subject's plasma Zn⁺⁺ levels relative to the level prior to treatment. An improved cognitive function may also occur.

Preferably, the metal binding agent results in the maintenance or decrease in reactive oxygen species levels in a subject being treated relative to the level prior to treatment, or relative to the level in an age-matched untreated subject.

Specific metal binding agents contemplated by the present invention include 8-hydroxy-quinolines or derivatives thereof, which have the ability to bind to zinc, copper or iron such as Zn²⁺, Cu²⁺ or Fe³⁺ ions and which exhibit one or more of the following characteristics:
(a) ability to inhibit Zn²⁺-, Cu²⁺- or Fe³⁺-induced amyloid aggregation in vitro;
(b) ability to cross the blood-brain barrier; and/or
(c) possess minimal or absent neurotoxicity in vitro.

One preferred bio-available copper/zinc metal binding agent is iodochlorhydroxyquin, also known as clioquinol (CQ). Preferably the metal binding agent also exhibits anti-oxidant activity.

The agents of the present invention which are capable of modulating the level of plasma zinc and/or of plasma copper while concomitantly decreasing the levels of reactive oxygen species and compositions comprising same may be used in the manufacture of a medicament for use in the treatment of neurological disorders and/or in arresting cognitive impairment such as pre- or mild cognitive impairment or memory loss. Such neurological disorders include, for example, neurological amyloidosis. The agents may be used systemically or locally such as topically.

The present invention contemplates, therefore, a method for the prophylactic and/or therapeutic treatment of a disorder or condition resulting from or exacerbated by oxidative stress and which comprises symptoms of cognitive impairment or memory loss, said method comprising administering to said subject an effective amount of an agent or a derivative, homolog, analog, chemical equivalent or mimetic thereof, which agent decreases the level of reactive oxygen species, resulting in the amelioration of symptoms of and/or otherwise arresting cognitive impairment such as pre- or mild cognitive impairment or memory loss.

The present invention further contemplates a method for the prophylactic and/or therapeutic treatment of mild cognitive impairment (MCI) or memory loss, said method comprising administering to said subject an effective amount of an agent or a derivative, homolog, analog, chemical equivalent or mimetic thereof, which agent modulates the level of reactive oxygen species and/or modulates the level of plasma zinc and/or copper.

In either of the above methods, the preferred agents are 8-hydroxyquinoline compounds such as those disclosed in Formula I herein.

### BRIEF DESCRIPTION OF THE FIGURES

FIGS. 1A and 1B are graphical representations showing the mean change in cognitive abilities over time (+SD) from baseline (as assessed with ADAS-cog) in (A) two arms of clioquinol vs placebo and (13) stratification by severity within treatment arms [less-severely affected (ADAS-Cog<25), more-severely affected (ADAS-cog>25) (*p<0.05; ** p<0.01)].

FIGS. 2A and 2B are graphical representations showing the mean change in plasma Aβ₄₂ levels over time (+SD) from baseline in (A) the arms of clioquinol vs placebo and (B) stratification by severity, as described for FIG. 1.

FIGS. 3A and 3B are graphical representations showing the mean change over time (+SD) from baseline in (A) plasma zinc and (B) plasma copper, in the two arms of clioquinol vs placebo.

### DETAILED DESCRIPTION OF THE PREFERRED EMBODIMENTS

The present invention provides agents and methods for the treatment and/or prophylaxis of disorders and/or conditions associated with oxidative stress and which have symptoms of cognitive impairment or memory loss. Such disorders or conditions are neurological disorders or conditions which include any neurological state which results in or otherwise contributes to cognitive impairment such as pre- or mild cognitive impairment or memory loss. Alternatively, or in addition, a neurological condition results from or is facilitated by an increase in the level of reactive oxygen species. Accordingly, the present invention provides agents and methods for the treatment of a condition associated with reactive oxygen species or other form of oxidated stress such as leading to a neurological condition. Preferred agents of the present invention are metal binding agents which sequester or bind to metal ions. One example is iodochlorhydroxyquin [PBT-1; also known as clioquinol (CQ)], which is a bio-available Cu/Zn binding agent.

In a preferred embodiment, levels of reactive oxygen species are generally decreased in subjects with mild to moderate cognitive impairment or mild to moderate neurological disease arising from oxidative stress.

Before describing the present invention in detail, it is to be understood that unless otherwise indicated, the subject invention is not limited to specific formulations of components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context clearly dictates otherwise. Thus, for example, reference to “an active agent” includes a single active agent, as well as two or more active agents; and so forth.

In describing and claiming the present invention, the following terminology is used in accordance with the definitions set forth below.

The expression “improving cognitive function” as used herein means slowing or arresting decline in cognitive function, increasing cognitive functioning, preventing or deferring the onset of cognitive dysfunction, relative to age-matched controls. Cognitive function may suitably be assessed by tests well known in the art, such as the ADAS-cog test, or other conventional cognitive screening tests, such as the Mini Mental Status Exam, and the Memory Impairment Screen. A more powerfully discriminating cognitive test, such as the CogState test (CogState Ltd, www.cogstate.com), may also be used.

The expression “oxidative stress” refers to the process whereby the amount of free radicals or reactive oxygen species increase, subsequent cell damage occurs and disease results. Free radicals are aggressive atoms or molecules that cause damage when they react with cell components. They are highly reactive due to unimpaired electrons. Free radicals attack the nearest stable molecule and sequester its electron, thereby oxidizing the molecule. Indicators of oxidative stress caused by free radicals include damaged DNA bases, protein oxidation products and lipid peroxidation products.

The term “metal binding agent” is used herein in its broadest sense, and refers to compounds having two or more donor atoms capable of binding to a metal atom, preferably copper, zinc or iron. In one particular embodiment such as in the treatment of AD, the metal binding agent will have a higher thermodynamic stability than that of the corresponding Aβ-metal ion complex. Preferred forms of copper, zinc and iron are Cu, Zn and Fe ions such as Cu²⁺, Zn²⁺ and Fe³⁺. The metals may be referred to herein by their full name or two letter abbreviation.

The term “a specific metal binding agent” as used herein refers to an 8-hydroxyquinoline or a derivative thereof which has the ability to bind to, for example, Zn²⁺, Cu²⁺ or Fe³⁺ ions.

The terms “compound”, “agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc.

Reference to a “compound”, “agent”, “pharmacologically active agent”, “medicament”, “active” and “drug” includes combinations of two or more actives such as one or more metal ion metal binding agents. A “combination” also includes a two-part or more such as a multi-part pharmaceutical composition where the agents are provided separately and given or dispensed separately or admixed together prior to dispensation.

As used herein, the term “therapeutically effective amount” means an amount of a compound of the present invention effective to yield a desired therapeutic response, for example to prevent or treat a disease which is susceptible to treatment by administration of a pharmaceutically-active agent. A “prophylactically effective amount” has a similar definition.

The specific “therapeutically effective amount” will of course vary with such factors as the particular condition being treated, the physical condition and clinical history of the subject, the type of animal being treated, the duration of the treatment, the nature of concurrent therapy (if any), and the specific formulations employed and the structure of the compound or its derivatives.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emusifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable. The carrier may be liquid or solid, and is selected with the planned manner of administration in mind.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a oxidative stress-mediated condition or symptom such as cognitive impairment such as pre- or mild cognitive impairment or memory loss.

The compound of the invention may be administered orally, topically, or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

Generally, the terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof, and/or may be therapeutic in terms of a partial or complete cure of a disease. The subject is generally a human. The present invention extends, however, to non-human primates or non-primates such as used in animal model testing.

“Treating” as used herein covers any treatment of, or prevention of disease, and includes preventing the disease from occurring in a subject which may be predisposed to the disease, but has not yet been diagnosed as having it; inhibiting the disease; i.e. arresting its development; or relieving or ameliorating the effects of the disease; i.e. causing regression of the effects of the disease.

The present invention provides, therefore, drugs and other agents which modulate the level of plasma zinc and/or copper or their ionic forms while concomitantly decreasing levels of free radicals or reactive oxygen species and/or maintaining or decreasing the levels of Aβ. Preferably, the agents are metal binding agents which sequester or bind to copper and zinc ions.

In a preferred embodiment, the administration of a metal binding agent results in a decrease of reactive oxygen species and/or the elevation of a subject's plasma zinc levels relative to their level prior to treatment. In addition or alternatively, the level of Aβ is maintained or decreased and/or there is an improvement in or an amelioration in the symptoms of cognitive impairment such as pre- or mild cognitive impairment or memory loss. Preferably, the subject's plasma copper levels do not change substantially relative to the level prior to treatment.

Subjects requiring treatment for neurological dysfunction frequently display a level of cognitive impairment such as pre- or mild cognitive impairment or memory loss, varying from moderate to severe. It is proposed herein that the agents of the present invention ameliorate the symptoms of cognitive impairment such as pre- or mild cognitive impairment or memory loss.

In a particularly preferred embodiment, the levels of free radicals or reactive oxygen species in subjects exhibiting moderate to severe cognitive impairment such as pre- or mild cognitive impairment or memory loss, are at least maintained relative to the level prior to treatment.

Particularly preferred metal binding agents in the context of the present invention include 8-hydroxyquinolines or derivatives, homologs, analogs or chemical equivalents or mimetic thereof which have the ability to bind to Zn²⁺, Cu²⁺ or Fe³⁺ ions and which further exhibit one or more of the following characteristics:—
(a) ability to decrease levels of reactive oxygen species;
(b) ability to inhibit Zn²⁺-, Cu²⁺- or Fe³⁺-induced amyloid aggregation in vitro;
(c) ability to cross the blood-brain barrier; and/or
(d) possess minimal or absent neurotoxicity in vitro.

Such 8-hydroxyquinolines are defined further below.

Preferably the agent also exhibits anti-oxidant activity. One particularly preferred agent in the context of the present invention is CQ.

Accordingly, another aspect of the present invention contemplates a method for the prophylactic and/or therapeutic treatment of a neurological condition characterized by impairment cognitive function including MCI, in a subject, said method comprising administering to said subject an effective amount of an agent or a derivative, homolog, analog, chemical equivalent or mimetic thereof, which agent decreases level of reactive oxygen species and/or modulates the level of plasma zinc and/or copper.

In another embodiment, the present invention further contemplates a method for the prophylactic and/or therapeutic treatment of mild cognitive impairment (MCI) or memory loss, said method comprising administering to said subject an effective amount of an agent or a derivative, homolog, analog, chemical equivalent or mimetic thereof, which agent decreases the level of reactive oxygen species and/or modulates the level of plasma zinc and/or copper.

The preferred agents are 8-hydroxyquinoline compounds described below.

In another embodiment, the compounds of the present invention may result in a maintenance or decrease in the level of Aβ. Reference herein to Aβ levels generally means plasma or serum Aβ levels.

Preferably, the prophylactic and/or therapeutic treatment results in slowing or arresting the decline in cognitive function in a subject suffering from a neurological disorder such as, for example, amyloidosis.

It may be advantageous for the agent to be able to be concentrated within the central nervous system. This capability may be designed into an agent through the inclusion of groups that enable the agent to be actively or passively transported into the brain, for example by formation of a lipophilic diester (See Australian Patent No. 739835).

It may also be advantageous for a single molecule of the agent to be able to provide three or more chelation points to enable a 1:1 ratio of agent:metal ion.

Accordingly, the present invention provides a method for the treatment and/or prophylaxis of a neurological condition or disorder associated with or exacerbated by oxidative stress which condition or disorder exhibits symptoms comprising cognitive impairment or memory loss, said method comprising administering to a subject in need thereof an effective amount of an agent of Formula I: 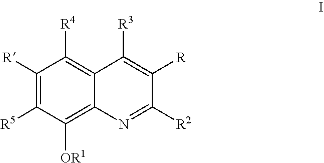
in which:
R¹ is H, optionally substituted alkyl, optionally substituted alkenyl, optionally substituted acyl, optionally substituted aryl, optionally substituted heterocyclyl, an antioxidant or a targeting moiety;
R² is H; optionally substituted alkyl; optionally substituted alkenyl; optionally substituted aryl; optionally substituted heterocyclyl; optionally substituted alkoxy; an antioxidant; a targeting moiety; COR⁶ or CSR⁶ in which R⁶ is H, optionally substituted alkyl, optionally substituted alkenyl, hydroxy, optionally substituted aryl, optionally substituted heterocyclyl, an antioxidant, a targeting moiety, OR⁷, SR⁷ or NR⁷R⁸ in which R⁷ and R⁸ are either the same or different and selected from H, optionally substituted alkyl, optionally substituted alkenyl, optionally substituted aryl or optionally substituted heterocyclyl; CN; CH₂NR⁹R¹⁰, HCNOR⁹ or HCNNR⁹R¹⁰ in which R⁹ and R¹⁰ are either the same or different and selected from H, optionally substituted alkyl, optionally substituted alkenyl, optionally substituted aryl or optionally substituted heterocyclyl; OR¹¹, SR¹¹ or NR¹¹R¹² in which R¹¹ and R¹² are either the same or different and selected from H, optionally substituted alkyl, optionally substituted alkenyl, optionally substituted aryl or optionally substituted heterocyclyl or together form optionally substituted heterocyclyl; or SO₂NR¹³R¹⁴ in which R¹³ and R¹⁴ are either the same or different and selected from H, optionally substituted alkyl, optionally substituted alkenyl, optionally substituted aryl or optionally substituted heterocyclyl; and
R³, R⁴, R⁵, R and R′ are either the same or different and selected from H, optionally substituted alkyl, optionally substituted alkenyl optionally substituted alkoxy, optionally substituted acyl, hydroxy, alkylamino, alkylthio, alkylsulphonyl, alkylsulphinyl, halo, SO₃H, amine, optionally substituted aryl, optionally substituted heterocyclyl, an anti-oxidant or a targeting moiety,
with the proviso that when R¹ to R³, R and R′ are H, then R⁴ is not Cl and R⁵ is not I,
salts, hydrates, solvates, derivatives, pro-drugs, tautomers and/or isomers thereof.

In a related embodiment, the present invention further contemplates a method for the prophylactic and/or therapeutic treatment of mild cognitive impairment (MCI) or memory loss, said method comprising administering to said subject an effective amount of an agent of Formula I: 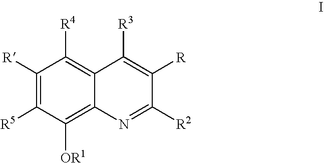
in which:
R¹ is H, optionally substituted alkyl, optionally substituted alkenyl, optionally substituted acyl, optionally substituted aryl, optionally substituted heterocyclyl, an antioxidant or a targeting moiety;
R² is H; optionally substituted alkyl; optionally substituted alkenyl; optionally substituted aryl; optionally substituted heterocyclyl; optionally substituted alkoxy; an antioxidant; a targeting moiety; COR⁶ or CSR⁶ in which R⁶ is H, optionally substituted alkyl, optionally substituted alkenyl, hydroxy, optionally substituted aryl, optionally substituted heterocyclyl, an antioxidant, a targeting moiety, OR⁷, SR⁷ or NR⁷R⁸ in which R⁷ and R⁸ are either the same or different and selected from H, optionally substituted alkyl, optionally substituted alkenyl, optionally substituted aryl or optionally substituted heterocyclyl; CN; CH₂NR⁹R¹⁰, HCNOR⁹ or HCNNR⁹R¹⁰ in which R⁹ and R¹⁰ are either the same or different and selected from H, optionally substituted alkyl, optionally substituted alkenyl, optionally substituted aryl or optionally substituted heterocyclyl; OR¹¹, SR¹¹ or NR¹¹R¹² in which R¹¹ and R¹² are either the same or different and selected from H, optionally substituted alkyl, optionally substituted alkenyl, optionally substituted aryl or optionally substituted heterocyclyl or together form optionally substituted heterocyclyl; or SO₂NR¹³R¹⁴ in which R¹³ and R¹⁴ are either the same or different and selected from H, optionally substituted alkyl, optionally substituted alkenyl, optionally substituted aryl or optionally substituted heterocyclyl; and
R³, R⁴, R⁵, R and R′ are either the same or different and selected from H, optionally substituted alkyl, optionally substituted alkenyl optionally substituted alkoxy, optionally substituted acyl, hydroxy, alkylamino, alkylthio, alkylsulphonyl, alkylsulphinyl, halo, SO₃H, amine, optionally substituted aryl, optionally substituted heterocyclyl, an anti-oxidant or a targeting moiety,
with the proviso that when R¹ to R³, R and R′ are H, then R⁴ is not Cl and R⁵ is not I,
salts, hydrates, solvates, derivatives, pro-drugs, tautomers and/or isomers thereof.

The present invention further contemplates use of an agent of Formula I in the manufacture of a medicament for the treatment and/or prophylaxis of a condition or disorder associated with or exacerbated by oxidative stress and with symptoms including cognitive impairment or memory loss.

Preferred agents of Formula I are as follows:— 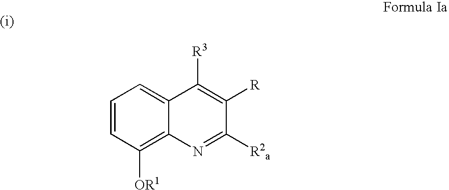
in which:
R, R¹ and R³ are as defined in Formula I above; and
R²a is H; optionally substituted C₁₋₆ alkyl; optionally substituted C₁₋₆ alkenyl; optionally substituted aryl; optionally substituted heterocyclyl; an antioxidant; a targeting moiety; COR⁶a or CSR⁶a in which R⁶a is H, optionally substituted C₁₋₆ alkyl, optionally substituted C₂₋₆ alkenyl, hydroxy, optionally substituted aryl, optionally substituted heterocyclyl or OR⁷a, SR⁷a or NR⁷aR⁸a in which R⁷a and R⁸a are either the same or different and selected from H, optionally substituted C₁₋₆ alkyl, optionally substituted C₂₋₆ alkenyl, optionally substituted aryl or optionally substituted heterocyclyl; CN; CH₂NR⁹aR¹⁰a, HCNOR⁹a or HCNNR⁹aR¹⁰ in which R⁹a and R¹⁰a are either the same or different and selected from H, optionally substituted C₁₋₆ alkyl, optionally substituted C₂₋₆ alkenyl, optionally substituted aryl or optionally substituted heterocyclyl; OR¹¹a, SR¹¹a or NR¹¹aR¹²a in which R¹¹a and R¹²a are either the same or different and selected from H, optionally substituted C₁₋₆ alkyl, optionally substituted C₂₋₆ alkenyl, optionally substituted aryl or optionally substituted heterocyclyl or together form optionally substituted heterocyclyl; or SO₂NR¹³aR¹⁴a in which R¹³a and R¹⁴a are either the same or different and selected from H or optionally substituted C₁₋₆ alkyl, optionally substituted C₂₋₆ alkenyl, optionally substituted aryl or optionally substituted heterocyclyl. 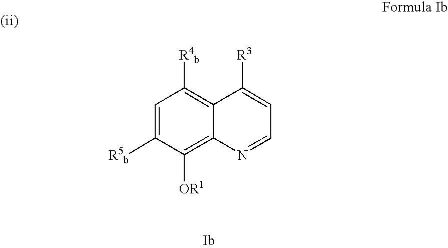
   in which:
R¹ and R³ are as defined in Formula I above;
R⁴b and R⁵b are either the same or different and selected from H; optionally substituted C₁₋₆ alkyl; optionally substituted C²⁻⁶ alkenyl; halo; an anti-oxidant; a targeting moiety, SO₃H; SO₂NR¹³aR¹⁴a in which R¹³a and R¹⁴a are as defined in Formula Ia above; or OR¹⁵b, SR¹⁵b or NR¹⁵bR¹⁶b in which R¹⁵b and R¹⁶b are either the same or different and selected from H, optionally substituted C₁₋₆ alkyl, optionally substituted C₂₋₆ alkenyl, optionally substituted C₁₋₆ acyl, optionally substituted aryl or optionally substituted heterocyclyl,
with the proviso that when R¹ and R³ are H, then R⁴b is not Cl and R⁵b is not I.

Preferred agents of Formula Ia are as follows: 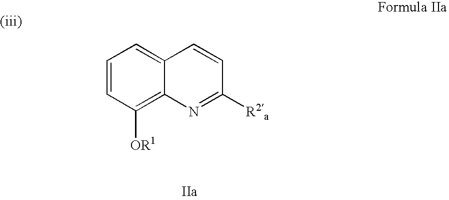
in which:
R¹ is as defined in Formula I above; and

R²′a is optionally substituted C₁₋₆ alkyl, optionally substituted C₂₋₆ alkenyl, optionally substituted aryl or optionally substituted heterocyclyl.

Formula IIa may represent agents in which an anti-oxidant moiety is attached to the C2 position of the 8-hydroxyquinoline in such a way that exposure to a pro-oxidative environment, that is, hydroxy radicals, will result in a molecule with enhanced metal chelation properties. 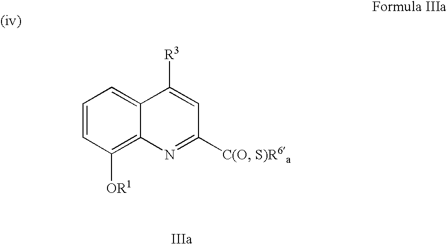
in which:
R¹ and R³ are as defined in Formula I above; and
R⁶′a is optionally substituted C₁₋₆ alkyl, optionally substituted C2-6 alkenyl, hydroxy, OR⁷′a, SR⁷′a, N₂R⁷′aR⁸′a or NR⁷′aR⁸′a in which R⁷′a and R⁸′a are either the same or different and selected from H, optionally substituted C₁₋₆ alkyl, optionally substituted aryl or optionally substituted heterocyclyl.

Formula IIIa represents agents in which a hydrophilic amide moiety is attached to the C2 position of the 8-hydroxyquinoline, so as to generally enhance solubility while maintaining membrane permeability. Agents of Formula IIIa also show enhanced metal chelation properties. 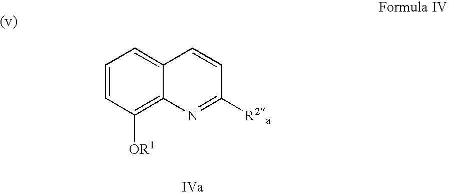
in which:
R¹ is as defined in Formula I above; and
R²″a is CN; CH₂NR⁹′aR¹⁰′a, HCNOR⁹′a or HCNNR⁹′aR¹⁰′a in which R⁹′a and R¹⁰′a are either the same or different and selected from H, optionally substituted C₁₋₆ alkyl, optionally substituted alkenyl, optionally substituted aryl or optionally substituted heterocyclyl.

Formula IVa represents agents that have improved metal chelation and optimized activity in the panel of assays described hereinafter. 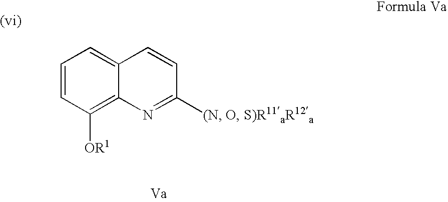
in which:
R¹ is as defined in Formula I above; and
R¹¹a′ and R¹²a′ are either the same or different and selected from H, optionally substituted C₁₋₆ alkyl, optionally substituted C₂₋₆ alkenyl, optionally substituted aryl and optionally substituted heterocyclyl or together form optionally substituted heterocyclyl. 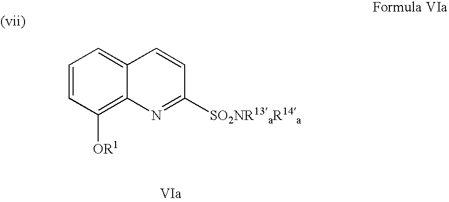
   in which:
R¹ is as defined in Formula I above; and
R¹³a′ and R¹⁴a′ are either the same or different and selected from H, optionally substituted C₁₋₆ alkyl, optionally substituted C₂₋₆ alkenyl, optionally substituted aryl or optionally substituted heterocyclyl.

Preferred agents of Formula Ib are as follows: 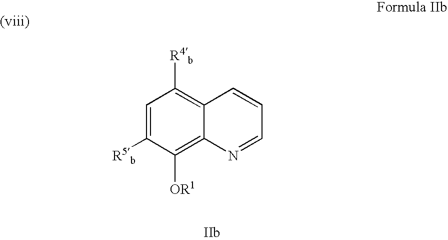
in which:
R¹ is as defined in Formula I above; and
R⁴b′ and R⁵a′ are either the same or different and selected from halo, C₁₋₆ alkyl, C₂₋₆ alkenyl, amine, SO₃H, optionally substituted aryl or optionally substituted heterocyclyl. 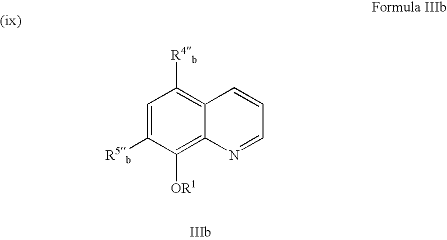
   in which:
R¹ is as defined in Formula I above;
R⁴b″ is H or halo; and
R⁵b″ is optionally substituted aryl or optionally substituted heterocyclyl. 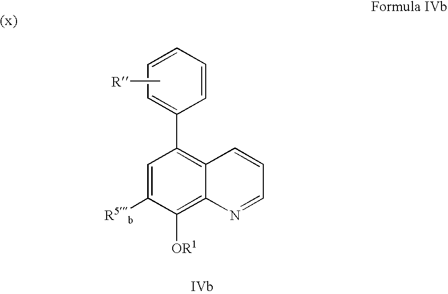
   in which:
R1 is as defined in Formula I above;
R″ is C₁₋₆ alkoxy, halo, C₁₋₆ alkyl, C₂₋₆ alkenyl or C₁₋₆ haloalkyl; and
R⁵b″ is H or halo. 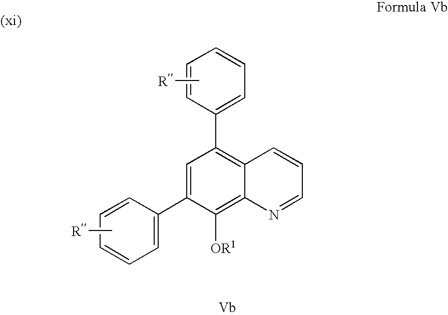
   in which
R¹ is as defined in Formula I above; and
R″ is as defined in Formula IVb above. 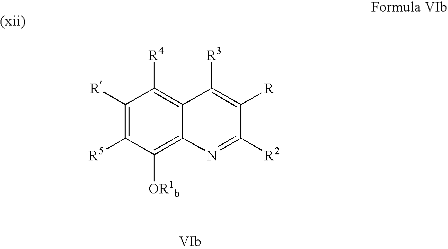
   in which:
R² to R⁵, R and R′ are as defined in Formula I above; and
R¹b″ is optionally substituted C₁₋₆ alkyl, optionally substituted aryl, optionally substituted aryl acyl, C₁₋₆ alkyl acyl or optionally substituted heterocyclyl.

Formula VIb represents agents in which the 8-hydroxyl group on the quinoline is blocked to form a pro-drug, in particular an ester pro-drug. The 8-hydroxy represents a principal site of metabolism for the agent of Formula I: conjugation with glucuronic acid or sulphate gives a hydrophilic species ready to be excreted. Such conjugates probably do not pass the blood brain barrier. The ester pro-drug may protect the agent of Formula I from conjugation. Esterases integral to the blood brain barrier may then release the C8-hydroxy on passage through that barrier activating the compound for its role in the central nervous system.

Agents which are particularly preferred, in view of their neurological activity, are shown below:

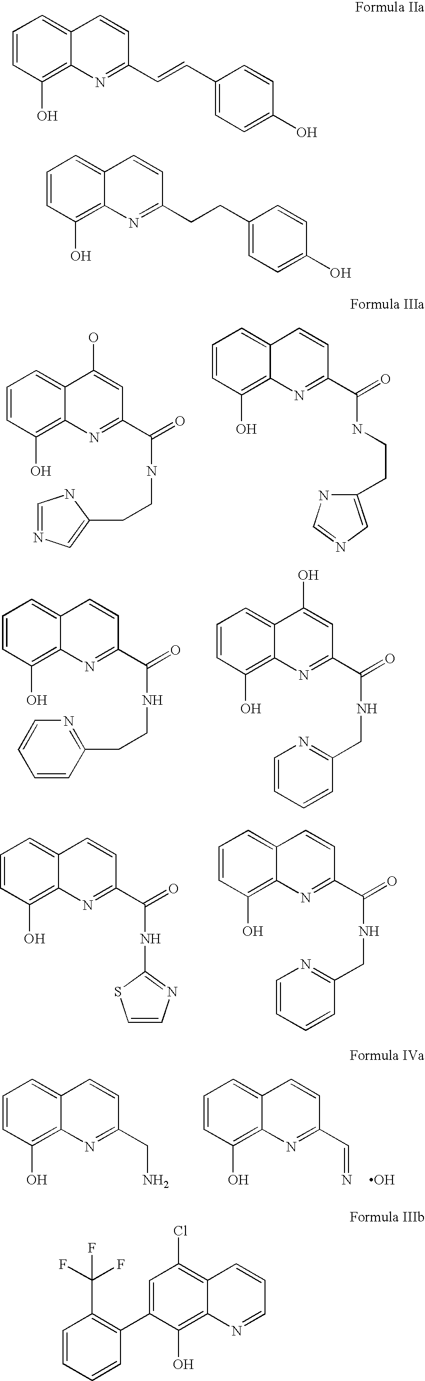

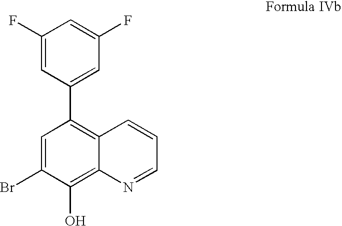

In preferred embodiments, the agents and methods of the present invention result in increased serum zinc levels, and/or decreased levels of plasma Aβ. Most preferably, the Aβ is Aβ₄₂. Preferably, serum copper levels are not affected.

Reference herein to “zinc”, “copper” and “iron” include their ionic forms such as but not limited to Zn⁺⁺, Cu⁺⁺ and Fe⁺⁺⁺.

The present invention also provides for the use of the agents, compounds and compositions disclosed herein as neurotherapeutic or neuroprotective agents, more preferably as anti-amyloidogenic agents, for the treatment and/or prophylaxis of a neurological condition and, in particular, one which involves a decline in cognitive function. Preferably, the neurological condition is neurological amyloidosis such as AD.

Further, the present invention contemplates the use of the agents, compounds and compositions disclosed herein in the manufacture of a medicament for use in the treatment and/or prophylaxis of a neurological condition, such as a neurological amyloidosis.

The term “neurological condition” is used herein in its broadest sense and refers to conditions in which various cell types of the nervous system are degenerated and/or have been damaged as a result of neurological disorders or injuries or exposures. In particular, compounds of Formula I or II can be used for the treatment of resulting conditions, in which damage to cells of the nervous system has occurred due to surgical interventions, infections, exposure to toxic agents, tumours, nutritional deficits or metabolic disorders. In addition, compounds of the Formula I or II can be used for the treatment of the sequelae of neurological disorders, such as AD, Parkinson's disease, multiple sclerosis, amylotrophic lateral sclerosis, epilepsy, drug abuse or drug addiction (alcohol, cocaine, heroin, amphetamine or the like), spinal cord disorders and/or injuries, dystrophy or degeneration of the neural retina (retinopathies) and peripheral neuropathies, such as diabetic neuropathy and/or the peripheral neuropathies induced by toxins.

The term “neurological disorder” as used herein refers to an abnormality in which neuronal integrity is threatened. Neuronal integrity can be threatened when neuronal cells display decreased survival or when the neurons can no longer propagate a signal.

A “neurological disorder” also includes MCI.

Neurological disorders that can be treated with the compounds of the present invention include acute intermittent porphyria; adriamycin-induced cardiomyopathy; AIDS dementia and HIV-1 induced neurotoxicity; AD; amylotrophic lateral sclerosis; atherosclerosis; cataract; cerebral ischaemia; cerebral palsy; cerebral tumour; chemotherapy-induced organ damage; cisplatin-induced nephrotoxicity; coronary artery bypass surgery; Creutzfeldt-Jacob disease and its new variant associated with “mad cow” disease; diabetic neuropathy; Down's syndrome; drowning; epilepsy and post-traumatic epilepsy; Friedrich's ataxia; frontotemporal dementia; glaucoma; glomerulopathy; hemochromatosis; hemodialysis; hemolysis; hemolytic uraemic syndrome (Weil's disease); hemorrhagic stroke; Hallerboden-Spatz disease; heart attack and reperfusion injury; Huntington's disease; Lewy body disease; intermittent claudication; ischaemic stroke; inflammatory bowel disease; macular degeneration; malaria; methanol-induced toxicity; meningitis (aseptic and tuberculous); motor neuron disease; multiple sclerosis; multiple system atrophy; myocardial ischaemia; neoplasia; Parkinson's disease; peri-natal asphyxia; Pick's disease; progressive supra-nuclear palsy; radiotherapy-induced organ damage; restenosis after angioplasty; retinopathy; senile dementia; schizophrenia; sepsis; septic shock; spongiform encephalopathies; subharrachnoid hemorrage/cerebral vasospasm; subdural hematoma; surgical trauma, including neurosurgery; thalassemia; transient ischaemic attack (TIA); traumatic brain injury (TBI); traumatic spinal injury; transplantation; vascular dementia; viral meningitis and viral encephalitis.

Additionally, compounds of the present invention may also be used to potentiate the effects of other treatments, for example, to potentiate the neuroprotective effects of brain derived nerve growth factor.

The invention is particularly directed to conditions which induce oxidative damage of the central nervous system, including acute and chronic neurological disorders such as traumatic brain injury, spinal cord injury, cerebral ischaemia, stroke (ischaemic and hemorragic), subharrachnoid hemorrage/cerebral vasospasm, cerebral tumur, AD, Creutzfeldt-Jacob disease and its new variant associated with “mad cow” disease, Huntington's disease, Parkinson's disease, Friedrich's ataxia, cataract, dementia with Lewy body formation, multiple system atrophy, Hallerboden-Spatz disease, diffuse Lewy body disease, amylotrophic lateral sclerosis, motor neuron disease and multiple sclerosis.

In all aspects of the present invention the neurological disorder is preferably characterized by neurological amyloidosis, in which neurological damage results from the deposition of amyloid. The amyloid may be formed from a variety of protein or polypeptide precursors including but not limited to Aβ, synuclein, huntingtin and prion protein.

Thus, the neurological disorder is preferably selected from the group consisting of neurological diseases arising from oxidative stress, sporadic or familial AD, dementia associated with Down's syndrome, amyotrophic lateral sclerosis, motorneuron disease, cataract, Parkinson's disease, Creutzfeldt-Jacob disease and its new variant associated with “mad cow” disease, Huntington's disease, dementia with Lewy body formation, multiple system atrophy, Hallerboden-Spatz disease, neurological disease resulting from oxidative stress due to stroke, cardiovascular assault and/or diabetes and diffuse Lewy body disease.

More preferably the neurological amyloidosis is an Aβ-related condition, such as AD or dementia associated with Down's syndrome or one of several forms of autosomal dominant forms of familial AD (reviewed in St George-Hyslop, 2000, supra). Most preferably the cognitive disorder is a neurological disorder associated with oxidative stress.

A “neurological disorder associated with oxidative stress” includes a condition caused or exacerbated by the generation of excess free radicals or reactive oxygen species.

In a particularly preferred embodiment of all aspects of the present invention, prior to treatment the subject has moderately or severely impaired cognitive function, as assessed by the AD Assessment Scale (ADAS)-cog test, for example, an ADAS-cog value of 25 or greater where less than a value of 25 is considered mild to moderate cognitive impairment such as pre- or mild cognitive impairment or memory loss.

In all aspects of the present invention, the binding agent is preferably a specific metal binding agent which has the ability to bind to metal ions such as Zn⁺⁺, Cu⁺⁺ or Fe^{30 +} ions. More preferably the specific binding agent is an 8-hydroxyquinoline or a derivative thereof as provided herein. Most preferably the metal binding agent is CQ.

In all aspects of the present invention the metal binding agent is preferably administered in a dosage range of 100 to 1,500 mg/day, more preferably 250 to 750 mg/day. This may optionally be administered in a divided dose. Furthermore, the dosage may be adjusted so that it is given per two days, per three days, per four days, per five days or per week or per month.

In addition to slowing or arresting the cognitive decline of a subject, the methods and agents of the present invention may also be suitable for use in the treatment or prevention of neurological disorders, or may be suitable for use in alleviating the symptoms of neurological disorders. The agents and compounds of the present invention may be able to provide at least a partial reversal of the cognitive decline experienced by patients. If administered to a subject identified as having an increased risk of or a predisposition to a neurological disorder or exhibiting pre-clinical manifestations of cognitive decline such as MCI, the methods and compounds disclosed herein may be able to prevent or delay the onset of clinical symptoms, in addition to slowing or reducing the rate of cognitive decline.

Dementias are usually not diagnosed until one or more warning symptoms have appeared. These symptoms constitute the MCI syndrome as defined by the American Academy of Neurology, and refers to the clinical state of individuals who have memory impairment, but who are otherwise functioning well, and who do not meet clinical criteria for dementia (Petersen et al., Neurology 56: 1133-1142, 2001). It is generally accepted that MCI is a precursor of many diseases associated with increased levels of free radicals such as AD and Parkinson's disease and may also be a precursor of dementias resulting from other pathological causes. Symptoms of MCI include:
(i) memory loss which affects job skills;
(ii) difficulty performing familiar tasks;
(iii) problems with language;
(iv) disorientation as to time and place (getting lost);
(v) poor or decreased judgement;
(vi) problems with abstract thinking;
(vii) misplacing things;
(viii) changes in mood or behaviour;
(ix) changes in personality; and/or
(x) loss of initiative.

MCI can be detected using conventional cognitive screening tests, such as the Mini Mental Status Exam, and the Memory Impairment Screen and neuropsychological screening batteries.

The compounds and compositions of the present invention may be administered by any suitable route and the person skilled in the art will readily be able to determine the most suitable route and dose for the condition to be treated. Dosage will be at the discretion of the attendant physician, and will depend on the nature and state of the condition to be treated, the age and general state of health of the subject to be treated, the route of administration, and any previous treatment which may have been administered.

Methods and pharmaceutical carriers for preparation of pharmaceutical compositions are well known in the art, as set out in textbooks such as Remington's Pharmaceutical Sciences, 20^{th} Edition, Williams & Wilkins, Pennsylvania, USA.

The carrier or diluent and other excipients will depend on the route of administration and, again, the person skilled in the art will readily be able to determine the most suitable formulation for each particular case.

The compound of the present invention may optionally be administered in conjunction with one or more other pharmaceutically active agents suitable for the treatment of the condition; i.e. it may be given together with, before, or after one or more such agents. For example, where the condition is a β-amyloid related condition, particularly AD, the compound may be used in conjunction with treatment with another agent, such as an inhibitor of the acetylcholinesterase active site, for example, phenserine, galantamine, or tacrine; an anti-oxidant, such as Vitamin E or Vitamin C; an anti-inflammatory agent, such as flurbiprofen or ibuprofen, optionally modified to release nitric oxide (for example, NCX-2216, produced by NicOx), or an oestrogenic agent such as 17-β-oestradiol. The agent may also be vitamin B12.

The present invention includes the use of various pharmaceutical compositions useful for ameliorating disease. The pharmaceutical compositions may be prepared by bringing an agent of the present invention and optionally one or more other pharmaceutically active agents, or combinations of an agent of the present invention and one or more other pharmaceutically active agents, into a form suitable for administration to a subject, using carriers, excipients and additives or auxiliaries.

Frequently-used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include anti-microbials, anti-oxidants, metal binding agents and inert gases. Other pharmaceutically acceptable carriers include aqueous solutions, non-toxic excipients, including salts, preservatives, buffers and the like, as described, for instance, in Remington's Pharmaceutical Sciences, 2000, supra and The British National Formulary 43^{rd} Ed. (British Medical Association and Royal Pharmaceutical Society of Great Britain, 2002; <http://bnf.rhn.net>). The pH and exact concentration of the various components of the pharmaceutical composition are adjusted according to routine skills in the art; see Goodman and Gilman's “The Pharmacological Basis for Therapeutics” (7^{th} Ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The present invention is further described by the following non-limiting Examples.

### EXAMPLE 1

### Fluorometric H₂O₂ Assay

A fluorometric assay is used to test for the ability of a test compound to inhibit hydrogen peroxide generation by Aβ in the presence of copper based on dichlorofluoroscein diacetate (DCF; Molecular Probes, Eugene Oreg.). The DCF solution (5 mM) in 100% dimethyl sulphoxide (previously purged with argon for 2 hr at 20° C.) is deacetylated in the presence of 0.25 M NaOH for 30 min and neutralized at pH 7.4 to a final concentration of 1 mM. Horseradish peroxidase (HRP) stock solution is prepared to 1 μM at pH 7.4. The reactions are carried out in PBS, pH 7.4 in a 96 well plate (total volume=250 μl/well). The reaction solutions contain Aβ 1-42 at concentrations which may be in the range 50 nM to 1 μM, copper-glycine chelate (Cu-Gly, prepared by adding CuCl₂ to glycine in the ratio of 1:6 and added to the Aβ in the proportion 2Cu-Gly:1 Aβ), reducing agents including dopamine (5 μM) or ascorbic acid, deacetylated DCF 100 μM, and HRP, 0.1 μM. 1-10 μM EDTA or another chelator or metal binding agent may also be present as a control for free copper, but is not required for the assay to function. The reaction mixture is incubated at 37° C. for 60 min. Catalase (4000 units/ml) and H₂O₂ (1-2.5 μM) standards in PBS pH 7.4 may be included as positive controls. Fluorescence is recorded using a plate reader with excitation and emission filters at 485 nM and 530 nM, respectively. H₂O₂ concentration may be established by comparing fluorescence with the H₂O₂ standards. Inhibition of Aβ H₂O₂ production is assayed by including a given concentration of test compound(s) in the test wells.

### EXAMPLE 2

### Neurotoxicity Assays

Primary Cortical Neuronal Cultures

Cortical cultures are prepared as previously described (White et al., J Neuroscience 18: 6207-6217, 1998). Embryonic day 14 BL6Jx129sv mouse cortices are removed, dissected free of meninges and dissociated in 0.025% w/v trypsin. Dissociated cells are plated in 24 well culture plates (Greiner GmbH, Austria) at a density of 2×10⁶ cells/mL in MEM with 10% v/v FCS and 10% v/v HS. Cultures are maintained at 37° C. in 5% v/v CO₂. Prior to experiments, the culture medium is replaced with MEM plus N2 supplements.

Primary Cerebellar Granule Neuronal Cultures

Cerebella from post-natal day 5-6 (P5-6) mice are removed and dissected free of meninges and dissociated in 0.025% w/v trypsin. Cerebellar granule neurons (CGN) are plated in 24 well culture plates at 350 000 cells/cm² in BME (Gibco BRL) supplemented with 10% w/v FCS, 2 mM glutamine and 25 mM KCl. Gentamycin sulphate (100 μg/mL) is added to all plating media and cultures are maintained at 37° C. in 5% v/v CO₂.

### EXAMPLE 3

### Assays for Cell Viability

MTT Assay for Cell Viability

Cell viability is determined using the MTT assay. Culture medium is replaced with 0.6 mg/mL MTT in control salt solution (Locke's buffer containing 154 mM NaCl, 5.6 mM KCl, 2.3 mM CaCl₂, 1.0 mM MgCl₂, 3.6 mM NaHCO₃, 5 mM HEPES and 5.6 mM glucose, pH 7.4) for 30 min. The MTT is removed and cells solubilized with dimethyl sulfoxide. 100 μL aliquots are measured with a spectrophotometer at 570 nm.

LDH Assay for Cell Viability

Cell death is determined from culture supernatants free of serum and cell debris using the lactate dehydrogenase (LDH) Cytotoxicity Detection Kit (Boehringer Ingelheim) according to the manufacturer's instructions.

### EXAMPLE 4

### Assay for Neurotoxicity at Low Aβ Concentration

Cortical cells are prepared following the protocol of White et al. (1998, supra), with the following modifications:—
(A) On the fourth to fifth day, the medium is changed for Neurobasal medium plus B27 but minus antioxidant;
(B) On the eighth to ninth day, the medium is replaced by medium containing test reagents, including Aβ (200-1000 nM), Cu-Gly (400-2000 nM) and dopamine (5-20 μM in PBS).

EDTA (10 μM in PBS) is included throughout to eliminate undesired reactions between free copper and dopamine. However, when testing new drugs, it is advisable not to include EDTA in the Aβ-Cu-Dopamine mixture. For controls, the dopamine volume is replaced with PBS 7.4; the Cu-Gly volume is replaced with water and the Aβ volume is replaced with water.

Aβ peptide solution is prepared by dissolving the peptide in water and centrifuge at 13,000 rpm, for 3-5 min. The supernatant is carefully harvested and its concentration measured by absorbance at 214 nm using the absorbance standard curve.

The following is the mixture sequence and example of approximate volumes of each compound:—

For a final volume of 1000 μL, the following sequence is carried out:

Aβ is added using 6.3 μL of Aβ stock (80 μM), to give a final concentration of 500 nM. Thereafter 10 μL of Cu-Gly stock (100 μM) is added to give a final concentration of 1000 nM. 68.7 μL of H₂O and 10 μL of EDTA 1 mM are added, to give a final concentration 10 μM of EDTA. 900 μL of Neurobasal medium plus B27 without antioxidant or Locke's buffer is then added and the solution is mixed. 5 μL of freshly made Dopamine stock (1 mM) is then added to give a final dopamine concentration 5 μM, and the solution is mixed again. The cell medium in each well of the culture is replaced with 250 μL of the mixture, and the cultures are incubated for 16-24 h (37° C.). Following incubation, each well is gently washed twice with Locke's buffer and then the Locke's buffer is replaced with Neurobasal medium (250 μL). Three empty wells are included as background controls.

25 μL of MTS stock is added to each well and incubated for 2-4 hrs at 37° C. The absorbance is then read at 490 nM.

### EXAMPLE 5

### Caspase Assay

To measure caspase activity in neuronal cultures, growth medium is removed, cells are washed twice with control salt solution (pH 7.4) and ice-cold cell extraction buffer is added directly to the cultures. The extraction buffer consists of 20 mM Tris (pH 7.4), 1 mM sucrose, 0.25 mM EDTA, 1 mM dithiothreitol (DTT), 0.5 mM PMSF, 1% v/v Triton X-100 (Tx-100) and 1 μg/mL of pepstatin and aprotinin. After incubation for 15 min on ice, the extraction buffer is removed, centrifuged for 5 min at 4° C. in a microcentrifuge and 100 μL of supernatant is added to each well of a 96 well plate. 100 μL of 200 μM substrate (either DEVD-pNA, VEID-pNA or IETD-pNA for caspases 3, 6 and 8, respectively) is added to each well to give a final concentration of 100 μM substrate. Plates are incubated at 37° C. for 2, 4, 6 or 24 hr and the absorbance is determined at a wavelength of 415 nm (Abs415). The absorbance reading is compared to a known standard of pNA alone.

### EXAMPLE 6

### Annexin V Assay

To determine the level of annexin V binding to cells, cultures are washed twice with control salt solution (pH 7.4) followed by the addition of annexin V-FITC at a concentration of approximately 0.5 μg/mL in control salt solution (pH 7.4). Propidium iodide (10 μg/mL) is also added to the cultures at the same time. Cells are incubated in the dark for 30 min at ambient temperature and subsequently washed three times with fresh control salt solution. Analysis of FITC fluorescence (ex. 488 nm, em. 510 nm) is determined using a Leica DMIRB microscope. Photographs are taken with a Leica MPS 60 camera attachment using ASA400 colour film and negatives are scanned into Adobe Photoshop v2.0.1.

### EXAMPLE 7

### Lipoprotein Oxidation Assay

Two different assays of metal-mediated lipid peroxidation can be utilized. The first assay involves measuring the oxidative activity of metallated proteins. This is determined by mixing dialyzed metallated or native protein (at designated concentrations) with 0.5 mg/mL LDL for 24 hr (37° C.). Lipid peroxidation (LPO) is measured using a lipid peroxidation assay kit (LPO 486, Oxis International Inc. Portland, Oreg.) as per kit instructions. The level of LPO is determined by comparing absorbance (486 nm) with LDL alone (100% LPO). The second assay is used to measure the LPO activity of native proteins in the presence of free, non-protein-bound Cu. This involves adding non-metallated peptides (140 μM) to 0.5 mg/mL LDL together with 20 μM Cu-gly and assaying for LPO as for the metallated proteins. The level of LPO is determined by comparing the absorbance (486 nm) with LDL+Cu-gly (100% LPO). As a negative control, LDL is also exposed to dialysed Cu-gly solutions comparable to those used to Cu-metallate the proteins.

### EXAMPLE 8

### Cytotoxicity Induced by Cu-Metallated Proteins

Proteins or synthetic peptides, such as Aβ or synuclein, are mixed with metal-glycine solutions at equimolar or two-fold metal to protein concentration. Metal-protein mixtures are incubated overnight at 37° C. and then extensively dialysed (24 hr against two changes of dH₂O (3 L/change) at room temperature) using mini-dialysis cups with a 3,500 kilodalton cut-off (Pierce, Rockford, Ill.). Dialysis of proteins against PBS pH 7.4 resulted in metallated proteins with identical activity to dH₂O dialysis.

To determine their neurotoxic effects, metallated proteins, native proteins or peptides are added to two day-old primary cortical neuronal cultures. The cultures are also exposed to Cu-gly (5 or 10 μM or LDL. Positive control cultures are treated with Cu-gly+LDL or the LPO product, 4-hydroxy-nonenol (HNE, Sigma Chemicals). Cultures are assayed for cell death using the lactate dehydrogenase (LDH) assay kit (Roche Molecular Biochemicals, Nunawading, Australia) according to the manufacturer's instructions.

### EXAMPLE 9

### Acridine Orange Assay for Aβ-Mediated Loss of Lysosomal Acidification

Cultured mouse cortical neurons are treated with Aβ1-42 (20 μM) for 16 hr and then stained with 5 mg/ml acridine orange (AO) for 5 min at 37° C. 15 min at 37° C. The AO-induced fluorescence is measured with a red filter on a fluorescence microscope. AO is a lysosomotropic weak base which accumulates in the endosomal/lysosomal compartments and displays orange fluorescence during incubation. AO is sequestered inside the lysosomes as long as there is a substantial proton gradient over the lysosomal membranes. Treatment of cells with Aβ1-42 disrupts the lysosomal membrane proton gradient and relocalizes AO into the cytosol, as indicated by the loss of orange fluorescence within 16-24 hr.

### EXAMPLE 10

### Brain Amyloid Solubilization Assay

This assay is performed in order to assess the ability of a test compound to mobilize Aβ from the insoluble to the soluble phase of an extract of tissue from post mortem human AD brain.

Up to 0.5 g of plaque-bearing cortex without meninges is homogenized using a DIAX 900 homogenizer (Heudolph and Co, Kelheim, Germany) or other suitable device for three 30-second periods at full speed in 2 ml of ice-cold phosphate-buffered saline, pH 7.4. To obtain the phosphate-buffered saline-extractable fraction, the homogenate is centrifuged at 100,000×g for 30 min and the supernatant removed. Supernatant, either freeze-dried and resuspended or in unconcentrated form, is dissolved in 2001 μl of Tris-Tricine sodium dodecyl sulfate (SDS) sample buffer pH 8.3 containing 8% w/v SDS, 10% v/v 2-mercaptoethanol. Aliquots (10 μl) are then boiled for 10 minutes before SDS-polyacrylamide gel electrophoresis. The insoluble fraction of the cortical samples is obtained by resuspending the initial pelleted sample in 1 ml of phosphate-buffered saline. A 50 μl aliquot of this suspension is then boiled in 200 ml of sample buffer as above.

Tris-Tricine polyacrylamide gel electrophoresis is performed by loading appropriately diluted samples on to 10% to 20% gradient gels (Novex, San Diego, Calif.) followed by transfer on to 0.2-μm nitrocellulose membrane (Bio-Rad, Hercules, Calif.). Aβ is detected by using monoclonal antibody W02, which detects residues 5 through 8, 17 (or another suitable antibody) in conjunction with horseradish peroxidase-conjugated rabbit anti-mouse IgG (Dako, Denmark) and visualized by using enhanced chemiluminescence (e.g. ECL; Amersham Life Science, Buckinghamshire, UK). Each gel includes three lanes containing 0.5, 1, and 2 ng of synthetic Aβ₄₀ (Keck Laboratory, Yale University, New Haven, Conn.) as reference standards.

Blot films are scanned by using a suitable imaging system such as the UVP gel documentation system and densitometry performed using suitable software, e.g. UVP Labworks. The dynamic range of the film/scanner is determined by using a step tablet (No. 911ST600, Kodak, Rochester N.Y.), a calibrated film exposed by the manufacturer to provide steps of known increasing intensity. The quantifiable range of signal intensity for densitometric analysis of the mono- and dimeric Aβ bands is based on the comparison with a curve obtained by scanning and densitometry of the step tablet. Samples in which the signal intensity is low after preliminary assay may be re-assayed by using synthetic standards of lower or higher concentration.

All samples are analyzed at least twice, and gel loadings and dilutions are adjusted to fit within the quantifiable region of the standard curve. The proportion of soluble to insoluble Aβ may be used to determine the efficiency of extraction of the test compound compared with the efficiency of a known compound, such as bathocuproine or clioquinol.

### EXAMPLE 11

### Metal Partitioning

To assay effects upon the partitioning of various metals, including zinc and copper, following extraction of brain tissue in the presence of a test compound, soluble and insoluble fractions from an extract of human brain tissue are prepared as for the amyloid solubilization assay. Metals in the two fractions are analyzed by inductively-coupled plasma mass spectrometry, following appropriate pretreatment with nitric acid and/or hydrogen peroxide where necessary.

### EXAMPLE 12

### Effect of Administration of Agents on Aβ Deposits in Transgenic Animals

Transgenic mouse models are available for a number of neurological disorders, including AD (Games et al., Nature 373(6514): 523-527, 1995; Hsiao et al., Science 274(5284): 99-102, 1996); Parkinson's disease (Nasliah et al., Science 287(5456): 1265-1269, 2000); familial amyotrophic lateral sclerosis (ALS) (Gurney et al., Science 264(5166): 1772-1775, 1994); Huntington's disease (Reddy et al., Nat. Genet. 20(2): 198-202, 1998) and Creutzfeld-Jakob disease (CJD) (Telling et al., Proc. Natl. Acad. Sci. USA 91(21): 9936-9940, 1994). These animal models are suitable for testing the methods of the invention.

Transgenic mice of the strain APP2576 (Hsiao et al., 1996, supra) are used. Twelve to 15 month old female mice are selected and divided into groups for treatment. At this age amyloid deposits are expected to be present. For studies on prophylaxis, younger animals may be used, for example, mice of eight to nine months old. Female mice are used because they generally develop amyloid deposits at an earlier age than males.

Agents are suspended in 0.05% carboxymethylcellulose and administered by gavage. For CQ a dose of 30 mg/kg body weight is effective (Cherny et al., Neuron 30: 665-676, 2001); the dosage for other agents is likely to vary, depending on the solubility and bioavailability of the individual agent. A dose of 10-100 mg/kg body weight is expected to be suitable in most cases, although for some agents the dose may be lower or higher than this range.

Mice are sacrificed at intervals, and their brains examined to determine whether the treatment decreases brain amyloid formation, and to identify the most effective administration protocol. The levels of soluble and insoluble Aβ in the brain and serum are determined using calibrated Western blots. The Aβ plaque burden in the brain is examined immunohistochemically.

Other mice in each group are tested over a period of up to eight months for cognitive performance, using a Morris water maze according to standard methods. The general health and well-being of the animals is also measured every day by a blinded operator, using a five point integer scale which subjectively rates a combination of features, including motor activity, alertness and general health signs.

### EXAMPLE 13

### Clinical Trial of Clioquinol for Treatment of AD

On the basis of the pre-clinical data, a Phase II clinical trial was prepared of CQ for the treatment of AD. Inclusion criteria for this Phase II study targeted a region on the ADAS-cog scale (20-45) where patients are moderately demented, still living at home, but anticipating an accelerated deterioration as part of the natural history of AD, over the next 12 months. Because the primary outcome was efficacy, a triple-blind design was chosen.

Several considerations drove the choice of dose. In previous studies on transgenic mice, doses of 20-30 mg/kg of CQ orally daily for five days per week were markedly effective at inhibiting Aβ accumulation after two to three months of treatment. The human equivalent dose of 1500-2250 mg/day is close to the prescribed antibiotic dose of CQ (600 mg po qid). However, this magnitude of dose, administered for months, would raise concerns about SMON toxicity.

As CQ is conjugated to glucuronide followed by renal excretion, there was some concern that blood levels in the elderly might be elevated by inefficient liver metabolism, constraining the proposed dose even further. Therefore, a cautious dose escalation design was chosen, to maximize the chance of detecting a change in outcome measures, while minimizing the risk of adverse effects. The starting dose of 3.3 mg/kg/day, assuming 75 kg average weight, was within the same order of magnitude of the effective dose in the transgenic mouse model, but only about one tenth of the antibiotic dose.

Since there were no data from the transgenic mouse study of the effectiveness of doses less than 20 mg/kg/day, it was proposed that a beneficial effect might require a longer period of treatment than the 9-12 week duration of the mouse study (Cherny et al., 2001, supra). Therefore, a trial length of 36 weeks at an average dose which was approximately one-third of what was effective in the transgenic mice was chosen. The final dose of 10 mg/kg/day was half of an effective dose in mice.

Thirty-six patients were randomized [18 placebo and 18 clioquinol (CQ)]. Per protocol analyses were conducted on 33 patients for analyses up to 24 weeks and 32 patients at 36 weeks. Groups were similar across most demographic, biological and clinical variables at baseline. The effect of treatment was statistically significant in the more-severely affected group (baseline ADAS-Cog≧25), but not the less-severely affected group (ADAS-Cog<25). The effect in the more severely-affected group was due to a substantial increase in ADAS-Cog scores with placebo and a negligible increase in the CQ group. Amongst the less-severely affected patients, only small increases in ADAS-Cog scores occurred in both placebo and CQ groups. Plasma Aβ₄₂ declined in the CQ group and increased in the placebo group. Plasma Zn levels rose by 30%. The drug was safe and well tolerated by participants.

### EXAMPLE 14

### Methods

Ethical Issues

In compliance with Australian Commonwealth and Victorian State laws concerning consent from individuals whose cognitive function may be impaired to the extent of being unable to make informed judgements or decisions, “Consent to Special Procedures” administered by the Victorian Civil and Administrative Tribunal was obtained for each participant. In addition, third party consent was obtained from all carers, in accordance with Victoria's Guardianship Laws. SMON was described in detail in the plain language statement, and discussed verbally with both patient and carer at the time of giving consent. As partially effective treatments are now available for AD, it was considered unethical to have the comparison group on placebo only; hence, both treatment groups were placed on donepezil for the duration of the study. The study was approved by the Royal Melbourne Hospital Research Foundation's Clinical Research and Ethics Committee.

Study Population

The study took place at the AD clinical trials unit of the Mental Health Research Institute of Victoria and at the Royal Melbourne Hospital.

Criteria for inclusion in the study were:—
(a) informed consent;
(b) a diagnosis of probable AD by NINCDS-ADRDA criteria (McKhann et al., Neurology 34: 939-944, 1984);
(c) AD Assessment Scale-Cognitive (ADAS-Cog) score of 20-45 inclusive (Rosen et al., Am. J. Psychiatry 141: 1356-1364, 1984);
(d) Mini Mental State Examination (MMSE) score of 10-24 inclusive (Folstein et al., J. Psychiatr. Res. 12: 189-198, 1975);
(e) on donepezil hydrochloride 5 mg or 10 mg for at least six months;
(f) relative or carer willing and able to support the trial;
(g) able to complete trial examinations; and
(h) primary sensorial functions intact.

All female patients were postmenopausal.

Patients were excluded if they had a history of allergy to CQ; history or clinical evidence of peripheral neuropathy or optic neuropathy; co-existing illnesses or past history which may have affected cognitive function or nerve conduction, including alcohol abuse or dependency; metabolic deficiencies (e.g. unstable thyroid dysfunction); infections with neurotrophic organisms such as syphilis, HIV, CMV, or EBV; current major depressive episode according to DSM-IV criteria; co-existing illnesses which might confound the adverse event profile, such as diabetes, untreated vitamin B12 or folate deficiency, ulcerative colitis, Crohn's disease, chronic diarrhoea, or multiple sclerosis; other co-existing medical conditions which might compromise the patient if s/he were to participate in a clinical trial, such as a neoplasm currently active or likely to recur (except non-melanoma skin cancer), history of immunosuppression, gastrointestinal malabsorption, hypertension (BP>180 mmHg systolic or >95 mmHg diastolic), cardiac failure (orthopnea, JVP>5 cm, or peripheral oedema requiring the prescription of loop diuretics), a history of stroke in the last 6 months or a Hachinski score ≧6, haemoglobin >20% below lower limit of normal range, raised white cell count (20% above reference range), neutropenia (white cell count <2.5), abnormal liver function tests (>50% above reference range), abnormal creatinine clearance (<75% of reference range), abnormal fasting blood glucose (>50% above upper limit of normal range), abnormal thyroid function (TSH or T4>20% outside reference range), or positive hepatitis A, B or C IgM.

The following factors were obtained at baseline to determine whether they correlated with outcome measures: age, sex, premorbid IQ (estimated from the National Adult Reading Test (NART)), years of education, serum donepezil hydrochloride and apolipoprotein E(ApoE) allotype.

Study Design

The study was a triple blind, placebo-controlled, randomized design. Thirty-six patients and their carers were recruited to participate, with patients being randomized to receive either CQ or placebo; there were 18 patients in each arm. The duration of the study was 36 weeks. CQ dosage was 125 mg twice daily from weeks 0-12, increased to 250 mg twice daily from weeks 13-24, and finally, 375 mg twice daily from weeks 25-36.

All patients had been treated with donepezil hydrochloride for at least six months prior to recruitment. The dose of donepezil was optimised by each patient's physician to maximize clinical benefit and to minimize side effects. This dose was maintained for the duration of the study, and patients were to be withdrawn from the study if, at regular review, the dose of donepezil required alteration for any reason.

The study medication and placebo were presented as enteric-coated capsules (125 mg were blue, 250 mg were brown), randomized in blocks of six. Presentation after increase to 250 mg twice daily was as 2×125 mg per dose; after increase to 375 mg, presentation was twice daily 1×125 mg and 1×250 mg per dose. This was to allow the dose to be reduced by 125 mg in each instance, i.e. to the previous dose, if the patient did not tolerate an increase in dose of study drug or placebo.

Study Procedures

Screening procedures consisted of a full medical history, full physical, neurological and ophthalmic examination, blood and urine tests and psychometric tests (ADAS-Cog, MMSE) to confirm the patient's eligibility for the study. Nerve conduction tests and visual evoked responses were conducted between the screening and baseline visits to provide a baseline measurement and to exclude patients with undiagnosed peripheral neuropathies or visual disturbances. Blood was collected for ApoE allotyping and determination of baseline plasma levels of CQ, metals and Aβ prior to randomization.

The study lasted 36 weeks, with 13 visits (including screening). Eligible subjects were randomized to receive either CQ or placebo. All patients continued their study entry dose of donepezil, and all patients received 100 μg vitamin B12 IM every four weeks.

Outcome Measures

The primary efficacy variable was a change in the baseline score on the AD Assessment Scale (ADAS), which was conducted at baseline and at weeks 4, 12, 24 and 36. This readout was chosen for comparability of treatment effects with current therapeutic agents, such as donepezil, for which efficacy trials also used ADAS as their primary outcome measure (Rogers et al., Neurology 50: 136-145, 1998). Although numerous neuropsychological tests could be considered as secondary measures, it was necessary to avoid fatiguing the subjects at review. Therefore the only other cognitive test performed was the Mini-Mental State Exam (MMSE), which is well characterized and easily implemented. The Clinician's Interview Based Impression of Change (CIBIC), a subjective observational index also used in efficacy trials of acetylcholinesterase inhibitors, was conducted at baseline and at weeks 12, 24 and 36 by an independent researcher who was not part of the study team. Blood samples for measurement of plasma Aβ and plasma zinc and copper were all taken four weekly, by antecubital fossa venepuncture.

Therapeutic Drug Monitoring

CQ drug assays were conducted over six hours at weeks 12, 24 and 36. The patient's blood was obtained via a heparinized indwelling catheter before the administration of CQ on these days, and then drawn again at 2, 4 and 6 hrs post dose. This was done to achieve pharmacokinetic data to correlate with other outcome measures.

Safety Measures

Standard adverse event reporting to a safety monitoring committee, consisting of physicians independent of the study, was conducted to review adverse events at three monthly intervals and on an emergent basis. Following baseline, safety visits were conducted at weeks 2, 4, 8, 12, 16, 20, 24, 26, 28, 32 and 36. The patient and carer were questioned about any changes which might have occurred in the patient's health or medications since the last visit. Standard biochemical, renal and liver function, full blood examination, serum vitamin B12 and folate levels, blood pressure and weight were documented at each visit. A neurological examination was conducted at each visit to assess for peripheral neuropathy and optic neuropathy, and visual evoked responses, nerve conduction studies and a full ophthalmic examination (visual acuity, colour vision, fundal examination and visual field) were conducted at screening, at week 18 and at two weeks after trial completion. An ECG was performed at baseline and at weeks 12 and 24.

Extension Study

All patients who completed the Phase II trial were invited to continue on a 48 week, prospective, open-label study of CQ. All were allocated to receive CQ 125 mg bid, increased after two weeks to 250 mg bid, then 375 mg BD at four weeks, while remaining on donepezil and vitamin B12. Patients who failed to tolerate dose increases beyond the 250 mg/day, 500 mg/day and 750 mg/day dosages in the blinded phase were placed on the highest tolerable dose beyond the previously achieved dose, at the clinical discretion of the investigator in the extension phase. Outcome and safety measures were the same as for the blinded phase. The length of the extension study was based upon an estimation of the time required to complete the blinded Phase II clinical trial, so that subjects would be able to continue to take the drug until they could be advised about the results of the trial.

Data Preparation and Statistical Analysis

Concealed randomization was conducted in blocks of six by the Institute of Drug Technology, a body which was independent of the study. An independent data monitoring company checked for omissions and validated entries in case report forms, and double entered the data into Microsoft Access©, completing validation and consistency checks. Before the analyses, each patient's randomization arm was labelled either ‘A’ or ‘B’. This ensured that the primary analyses were conducted blind to the subjects' randomization group, and hence were triple blind).

Two-way analysis of variance and covariance was used to analyse the major outcome variables with group (treatment versus placebo) as a between-subjects factor and occasion (baseline versus subsequent measurement occasions) as a within-subjects factor. Evidence for efficacy was indicated by a significant group by occasion interaction. Differences between groups on categorical measures were analyzed using exact statistical methods in order to maximize power.

The influence of confounding variables was controlled using analysis of covariance and linear regression models where appropriate. Based on the assumption of 50% shared variance between measurement occasions (i.e. r=0.70), power to detect an effect of one standard deviation difference in change between groups from baseline to week 36 would have been approximately 80% if 15 subjects were recruited per group. Since an attrition rate of 15% has been observed in similar populations, 18 patients were recruited into each arm.

The design also included a subset analysis of outcome measures, in which the cohort was divided into two equal size groups by the median ADAS-Cog score at baseline, yielding a less severely-affected subset, and a more severely-affected subset.

### EXAMPLE 15

### Results

Subject Recruitment and Demographics

Thirty-six subjects were recruited over a 12 month period, commencing April 2000. Of these, 33 completed the study. Two subjects were lost from each arm. In the placebo arm, one patient died and the other withdrew because of an illness unrelated to AD. In the treatment arm, one subject withdrew because of behavioural changes associated with AD (paranoia, non-cooperation, refusal to be tested). One subject was not included in the analysis, because the initial diagnosis of AD was probably incorrect; the symptoms and signs evolved into a picture characteristic of Diffuse Lewy Body disease.

The groups did not differ across any of the demographic, biological and clinical parameters at baseline, other than the treatment arm scoring lower (p=0.02) on the test for premorbid IQ (derived from the NART). The NART was subsequently entered into analysis as a co-variate, and was found not to be significant on any occasion.

Effect on Cognitive Decline

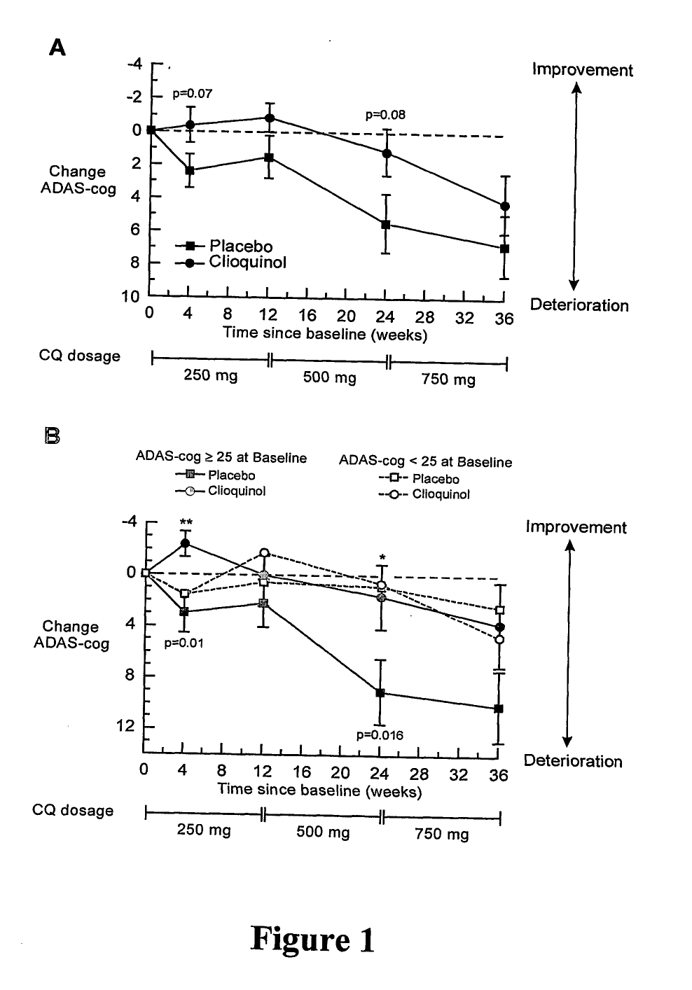

The primary clinical outcome of efficacy, as judged by ADAS-Cog, showed a trend between the two arms. In FIG. 1A, the trend indicates a slowing in the rate of decline of cognitive function. Stratification of the population into less- or more-affected around the median value of the baseline ADAS-Cog (values <25, ≧25) demonstrated that CQ had a significant effect on preventing cognitive decline in the more severely-affected group. This is illustrated in FIG. 1B.

Effect on Plasma Aβ

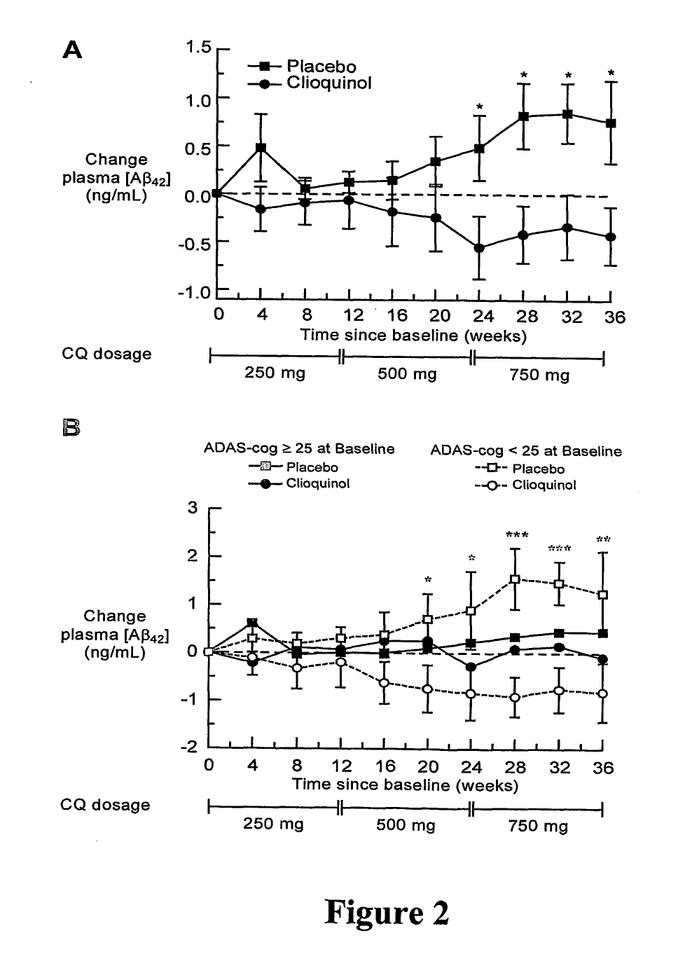

Measurement of plasma Aβ₄₂ showed a very significant lowering in the CQ-treated group from week 20 onwards; over the same time, plasma Aβ₄₂ in the placebo group increased. This is illustrated in FIG. 2A. These changes were more evident in the less-severely affected individuals, as shown in FIG. 2B, in whom the absolute levels of Aβ42 tended to be higher than in the more-severely affected group.

Analysis of plasma Aβ₄₀ levels showed overall similar trends, with significant differences between placebo and CQ groups observed at weeks 8, 32, and 36 in the less-severely affected groups.

Effect on Plasma Zn and Cu

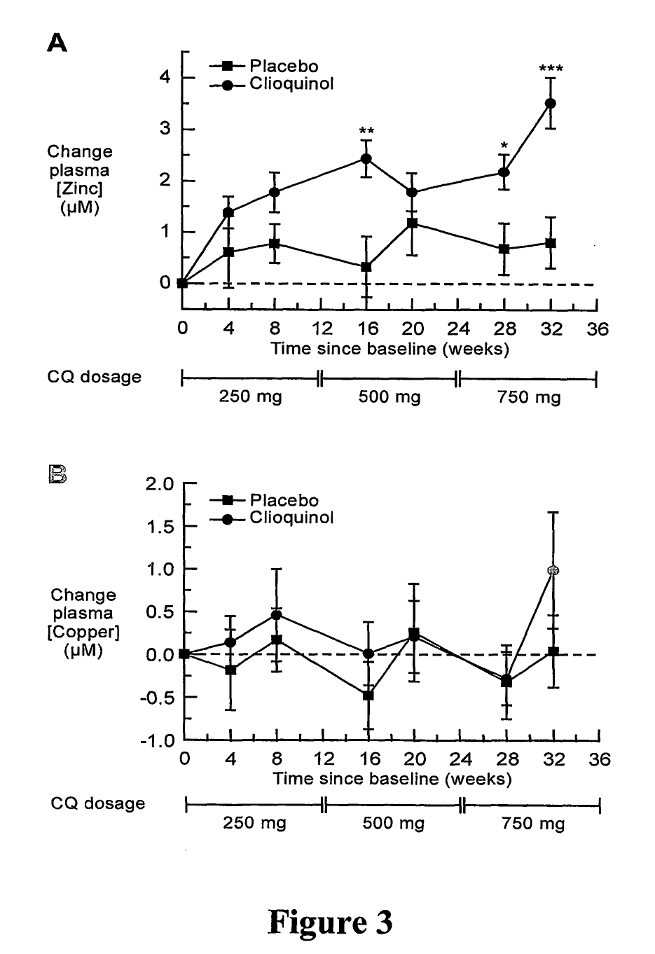

Administration of CQ was associated with a significant elevation of total plasma Zn, as shown in FIG. 3A, but with no effect on plasma Cu, as shown in FIG. 3B. Mean absolute levels of Cu (13.1 μM/l) were within the age-related normal range (Rahil-Khazen et al., Clin. Chem. Lab. Med. 38(8): 765-772, 2000).

Blood Levels of CO

The steady state (basal) levels of CQ at total daily dosages of 250, 500 and 750 mg were 4.03±2.10, 6.74±3.70, 7.60±2.15 μg/ml, respectively, and did not show significant correlations with ADAS-Cog results, metal levels or Aβ levels.

Safety Results and Analysis

There were a total of 131 attributable events reported, 61 in the treatment group and 50 in the placebo group. This mean number of discrete events per subject was not significantly different between arms. The results are summarized in Table 1. Five patients developed a serious adverse event (SAE). Four non-attributable SAEs were recorded. There was one death due to intracranial hemorrhage (placebo) and three hospitalizations for hip pain (placebo), syncope due to impaired cardiac function (CQ) and confusion (placebo).

TABLE 1
Attributable adverse events with a risk of greater
than 10% in either arm or where point estimate risk
ratio is greater than 2.0 or less than 0.5

| | Treatment | Placebo | Relative Risk |
|---|---|---|---|
| | (n = 18) | (n = 18) | (95% CI) |
| Cardiovascular | | | |
| Postural Hypotension | 12 | 11 | 1.09 |
| | | | (0.67, 1.79) |
| Postural Tachyardia | 12 | 8 | — |
| Postural Dizziness | 7 | 3 | 2.33 |
| | | | (0.71, 7.63) |
| Subjects with +1 postural | 13 | 14 | 0.93 |
| symptom | | | (0.64, 1.36) |
| Neurological | | | |
| Impaired nerve conduction | 3 | 1 | 3.0 |
| | | | (0.34, 26.2) |
| Impaired reflexes | 1 | 2 | 0.5 |
| | | | (0.05, 5.04) |
| Numb legs | 2 | 0 | — |
| Subjects with =1 neurological | 6 | 4 | 1.5 |
| symptom | | | (0.51, 4.43) |
| Gastrointestinal | | | |
| Diarrhoea | 1 | 4 | 0.25 |
| | | | (0.03, 2.02) |
| Constipation | 2 | 0 | — |
| Nausea | 2 | 0 | — |
| Abdominal pain | 2 | 1 | 2.0 |
| | | | (0.2, 20.1) |
| Subjects with =1 GI symptom | 5 | 4 | 1.25 |
| | | | (0.4, 3.91) |
| Genitourinary | | | |
| Microalbuminurea | 5 | 5 | 1.00 |
| | | | (0.35, 2.87) |
| Haematological | | | |
| Lymphopoenia | 0 | 3 | |
| Liver Function Tests | | | |
| Raised γGT | 2 | 1 | 2.0 |
| | | | (0.2, 20.1) |
| Raised bilirubin | 2 | 0 | — |
| Subjects with =1 abnormal LFT | 4 | 1 | 4.0 |
| | | | (0.49, 32.4) |
| Other | | | |
| Decreased vitamin B12 | 0 | 2 | — |
| Mean number discrete Adverse | 3.38 | 2.78 | Mean diff |
| Events | (61/18) | (50/18) | (95% CI): |
| (SD) Per subject | (2.14) | (1.48) | 0.611 |
| | | | (−0.64, 1.89) |
| | | | p = 0.327 |

Cardiac Safety

Symptoms of postural cardiac insufficiency were common, with 27/36 (75%) of subjects reporting at least one symptom of this nature. There were no significant between-group differences, as shown in Table 1.

Gastrointestinal Safety

Patients on CQ were less likely to develop diarrhoea, but more likely to demonstrate changes in liver function tests (LFT). This difference was also observed in group LFT changes. γ-GT, AST, ALT and bilirubin showed a small significant elevation and albumin fell significantly in the CQ arm. This was reflected by significant between-group differences in γ-GT and ALT changes. No subject developed any overt symptoms or signs of impaired liver function, and changes noted at week 24 were returning to normal by week 36.

Haematological Safety

There were no adverse haematological events in the CQ-treated subjects. However, there was a significant reduction in haemoglobin noted at weeks 24 (6.88 g/dl; 2.73, 11.0, p=0.003) and week 36 (5.5 g/dl; 0.72, 10.27. p=0.03) in the CQ arm. The difference at week 24 was significant between groups (F=6.135, P=0.02). The nature of the clinically irrelevant decrease in hemoglobin was uncertain, although there was no change to the mean cell volume, and no decrease in vitamin B12 or serum folate levels in either group.

Neurological Safety

The results of neurological tests are summarized in Table 2. Neurological symptoms or signs were uncommon. Subjects on CQ were at increased risk of displaying abnormal nerve conduction, but detailed analyses of nerve conduction data revealed that although calf and wrist latency significantly decreased in both arms by week 36, there were no significant between group differences in any nerve conduction parameter. There were no significant differences in the risk of impairment in visual acuity, colour vision, visual fields or fundoscopic abnormalities between the two arms of the study, as assessed by McNemar's test.

TABLE 2
Within-group changes to peripheral nerve latency from baseline to weeks 20 and 36

| | | | | | 95% | | | | |
|---|---|---|---|---|---|---|---|---|---|
| | | | | | Confidence | | | | |
| | | | | | interval of the | | | | |
| | | | Std | Std. Error | Difference | | | | Sig. |
| | | Mean | Dev. | Mean | Lower | Upper | t | df | (2-tailed) |
| Pair 1 | calf latency | .5750 | .5310 | .1328 | .2920 | .8580 | 4.331 | 15 | .001 |
| | (baseline)-calf | | | | | | | | |
| | latency (week 20) | | | | | | | | |
| Pair 2 | calf latency | .7937 | .6308 | .1577 | .4576 | 1.1299 | 5.033 | 15 | .000 |
| | (baseline)-calf | | | | | | | | |
| | latency (week 36) | | | | | | | | |
| Pair 3 | wrist 1 latency | .5824 | .8925 | .2165 | .1235 | 1.0412 | 2.690 | 16 | .016 |
| | week 1-wrist 1 | | | | | | | | |
| | latency week 20 | | | | | | | | |
| Pair 4 | wrist 1 latency | .7875 | .8277 | .2069 | .3464 | 1.2286 | 3.806 | 15 | .002 |
| | week 1-wrist 1 | | | | | | | | |
| | latency week 36 | | | | | | | | |
| Pair 5 | wrist 2 latency | 1.000E−01 | .3229 | 8.338E−02 | −7.8834E−02 | .2788 | 1.199 | 14 | .250 |
| | week 1-wrist 2 | | | | | | | | |
| | latency week 20 | | | | | | | | |
| Pair 6 | wrist 2 latency | .2429 | .4327 | .1157 | −6.9920E−03 | .4927 | 2.100 | 13 | .056 |
| | week 1-wrist 2 | | | | | | | | |
| | latency week 36 | | | | | | | | |

Randomization Drug = Clioquinol

| | | | | | | | | | |
|---|---|---|---|---|---|---|---|---|---|
| Pair 1 | calf latency | .7467 | .6556 | .1693 | .3836 | 1.1097 | 4.411 | 14 | .001 |
| | (baseline)-calf | | | | | | | | |
| | latency (week | | | | | | | | |
| | 20) | | | | | | | | |
| Pair 2 | calf latency | .9867 | .4673 | .1207 | .7279 | 1.2455 | 8.177 | 14 | .000 |
| | (baseline)-calf | | | | | | | | |
| | latency (week | | | | | | | | |
| | 36) | | | | | | | | |
| Pair 3 | wrist 1 latency | .3313 | .8677 | .2169 | −.1311 | .7936 | 1.527 | 15 | .148 |
| | week 1-wrist 1 | | | | | | | | |
| | latency week 20 | | | | | | | | |
| Pair 4 | wrist 1 latency | .5063 | .8668 | .2167 | 4.438E−02 | .9681 | 2.336 | 15 | .034 |
| | week 1-wrist 1 | | | | | | | | |
| | latency week 36 | | | | | | | | |
| Pair 5 | wrist 2 latency | 1.250E−02 | .2705 | 6.762E−02 | −.1316 | .1566 | .185 | 15 | .856 |
| | week 1-wrist 2 | | | | | | | | |
| | latency week 20 | | | | | | | | |
| Pair 6 | wrist 2 latency | .1688 | .5003 | .1251 | −9.7837E−02 | .4353 | 1.349 | 15 | .197 |
| | week 1-wrist 2 | | | | | | | | |
| | latency week 36 | | | | | | | | |

Randomization drug = placebo

Without limitation to the scope of the invention, the peak at four weeks may indicated that an even greater improvement in cognitive function may be achieved using higher dosage ranges.

The present longitudinal study is the first which has followed affected individuals over an extended period and disclosed a progressive decrease or maintenance in plasma Aβ levels. It is also the first to show that a specific metal binding agent can elevate and thereby restore plasma zinc levels to normal age-matched values.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Beyreuther K, Christen Y, Masters C L (eds) Neurological Disorders: Loss of Function Through Gain of Function. Springer. Berlin. 2001.
Bush A I, Masters C L. Clioquinol's return. Science 292: 2251-2252, 2001.
Cherny R A, Atwood C S, Xilinas M E et al. Treatment with a copper-zinc chelator markedly and rapidly inhibits β-amyloid accumulation in Alzheimer's disease transgenic mice. Neuron 30: 665-676, 2001.
Folstein M F, Folstein S E, McHugh P R. Mini-mental state: a practical method for grading the cognitive state of patients for the clinician. J. Psychiatr. Res. 12: 189-198, 1975.
Games, D; Adams, D; Alessandrini, R; Barbour, R; Berthelette, P; Blackwell, C; Carr, T; Clemens, J; Donaldson, T; Gillespie, F. Alzheimer-type neuropathology in transgenic mice over-expressing V717F beta-amyloid precursor protein. Nature. 373(6514): 523-527, 1995.
Goodman and Gilman's “The Pharmacological Basis for Therapeutics” (7^{th} Ed., 1985).
Gurney, M E; Pu, H; Chiu, A Y; Dal Canto, M C; Polchow, C Y; Alexander, D D; Caliendo, J; Hentati, A; Kwon, Y W; Deng, H X. Motor neuron degeneration in mice that express a human Cu,Zn superoxide dismutase mutation. Science 264(5166): 1772-1775, 1994.
Hsiao, K., Chapman, P., Nilsen, S., Eckman, C., Harigaya, Y., Younkin, S., Yang, F., Cole, G. Correlative memory deficits, Aβ elevation, and amyloid plaques in transgenic mice. Science 274(5284): 99-102, 1996.
Masliah, E; Rockenstein, E; Veinbergs, I; Mallory, M; Hashimoto, M; Takeda, A; Sagara, Y; Sisk, A; Mucke, L. Dopaminergic loss and inclusion body formation in alpha-synuclein mice: implications for neurological disorders. Science 287(5456): 1265-1269, 2000.
McKhann G, Drachman D, Folstein M F, Katzman R, Price D, Stadlen E. Clinical diagnosis of Alzheimer's disease: Report of the NINCDS-ADRDA work group under the auspices of the Department of Health and Human Services Task Force on Alzheimer's Disease. Neurology 34: 939-944, 1984.
Petersen, R. C, Stevenas, J. C., Ganguli, M., Tangalos, E. G., Cummings, J. L., and DeKosky, S. T. Practice parameter: Early detection of dementia: Mild cognitive impairment. Neurology 56: 1133-1142, 2001.
Rahil-Khazen R, Bolann B J, Ulvik R J. Trace element reference values in serum determined by inductively coupled plasma atomic emission spectrometry. Clin Chem Lab Med; 38 (8): 765-772, 2000.
Reddy, P H; Williams, M; Charles, V; Garrett, L; Pike-Buchanan, L; Whetsell, W O Jr; Miller, G; Tagle, D A. Behavioural abnormalities and selective neuronal loss in HD transgenic mice expressing mutated full-length HD cDNA. Nat Genet. 20(2): 198-202, 2998.
Remington's Pharmaceutical Sciences, 20^{th} Edition, Williams & Wilkins, Pennsylvania, USA.
Rogers S L, Farlow M R, Doody R S, Mohs R, Friedhoff L T. A 24-week, double-blind, placebo-controlled trial of donepezil in patients with Alzheimer's disease. Donepezil Study Group. Neurology 50: 136-145, 1998.
Rosen W G, Mohs R C, Davis K L. A new rating scale for Alzheimer's disease. Am J Psychiatry 141: 1356-1364, 1984.
Selkoe, D. J. Alzheimer's disease: genes, proteins and therapy. Physiol Rev 81(2): 741-766, 2001.
St George-Hyslop, P. H. Molecular genetics of Alzheimer's disease. Biol. Psychiatry 47: 183-199, 2000.
Telling, G C; Scott, M; Hsiao, K K; Foster, D; Yang, S L; Torchia, M; Sidle, K C; Collinge, J; DeArmond, S J; Prusiner, S B. Transmission of Creutzfeldt-Jakob disease from humans to transgenic mice expressing chimeric human-mouse prion protein. Proc. Natl. Acad. Sci. USA 91(21): 9936-9940, 1994.
White et al., J Neuroscience 18: 6207-6217, 1998.

1-39. (canceled)

40. A method for the treatment of Huntington's Disease (HD) or symptoms thereof in a subject, said method comprising administering to said subject an effective amount of Clioquinol (CQ) or a pro-drug thereof or a salt of CQ, wherein said pro-drug or said salt of CQ retains the ability to ameliorate the symptoms of HD.

41. The method of claim 40, wherein the symptoms comprise cognitive impairment or memory loss.

42. The method of claim 40 or 41, wherein said CQ or pro-drug or CQ salt, is administered in conjunction with at least one pharmaceutically acceptable compound used for treating a neurological disorder.

43. The method of claim 42, wherein said compound is selected from the group consisting of phenserine, galantamine, tacrine, Vitamin E, Vitamin C, flurbiprofen, ibuprofen, NCX-2216, 17-β-oestradiol, and vitamin B12.

44. The method of claim 40 or 41 wherein CQ is administered.

45. The method of claim 42 wherein CQ is administered.

46. The method of claim 45 wherein said compound is selected from the group consisting of phenserine, galantamine, tacrine, Vitamin E, Vitamin C, flurbiprofen, ibuprofen, NCX-2216, 17-β-oestradiol, and vitamin B12.

47. The method of claim 40 or 41, wherein the effective amount of CQ or pro-drug or CQ salt is in a dosage range of 100 to 1,500 mg/day.

48. The method of claim 40 or 41, wherein the effective amount of CQ or pro-drug or CQ salt is in a dosage range of 250 to 750 mg/day.

49. The method of claim 42, wherein the effective amount of CQ or pro-drug or CQ salt is in a dosage range of 100 to 1,500 mg/day.

50. The method of claim 42, wherein the effective amount of CQ or pro-drug or CQ salt is in a dosage range of 250 to 750 mg/day.

51. The method of claim 43, wherein the effective amount of CQ or pro-drug or CQ salt is in a dosage range of 100 to 1,500 mg/day.

52. The method of claim 43, wherein the effective amount of CQ or pro-drug or CQ salt is in a dosage range of 250 to 750 mg/day.
